(19) United States
(12) Patent Application Publication
(10) Pub. No.: US 2021/0228864 A1
(43) Pub. Date: Jul. 29, 2021

(54) ELECTRONIC DEVICES, ELECTRODES THEREOF, AND METHODS FOR PRODUCING THE SAME

(71) Applicant: Purdue Research Foundation, West Lafayette, IN (US)

(72) Inventors: Hyowon Lee, West Lafayette, IN (US); Hyunsu Park, Cambridge, MA (US); Zhihong Chen, West Lafayette, IN (US)

(21) Appl. No.: 17/143,491

(22) Filed: Jan. 7, 2021

Related U.S. Application Data
(60) Provisional application No. 62/958,485, filed on Jan. 8, 2020.

(51) Int. Cl. A61N 1/05; H01B 13/32; H01B 5/00; A61N 1/36; A61N 1/04

(52) U.S. Cl. CPC A61N 1/0529; H01B 13/32; H01B 5/002; H01B 5/14; A61N 1/36082; A61N 1/0472; A61N 1/36025

(57) ABSTRACT

Electronic devices having one or more platinum-based electrodes and methods of producing the same. Such an electronic device includes a platinum-based electrode having a protective layer thereon that includes graphene in an amount effective to reduce platinum corrosion of the electrode.

### CROSS REFERENCE TO RELATED APPLICATIONS

This application claims the benefit of U.S. Provisional Application No. 62/958,485, filed Jan. 8, 2020, the contents of which are incorporated herein by reference.

### BACKGROUND OF THE INVENTION

The present invention generally relates to electronic devices and electrodes thereof, including but not limited to neurostimulation devices and platinum electrodes thereof.

In general, invasive (implantable) and noninvasive neurostimulation devices (which encompasses what are referred to herein as neural interface devices) are electronic devices that have been used to target specific deep subcortical, cortical, spinal, cranial, or peripheral nerve structures to modulate neuronal activity, providing therapeutic effects for a myriad of neuropsychiatric disorders. Platinum (Pt) is widely used in neurostimulation devices as the preferred material for the electrodes of these devices. However, a well-known problem of using Pt, especially for a high-density neural interface device with microscale electrodes (referred to herein as microelectrodes), is that it can undergo irreversible electrochemical reactions during neurostimulation that can physically alter the electrode surface. Irreversible Pt corrosion can occur during neurostimulation due to cyclic formation and reduction of a platinum oxide (PtO₂) layer on the surface of a Pt electrode. Moreover, Pt can react with chloride ions during the anodic phases to form platinum chloride species that can affect cellular physiology. Both conditions can be particularly detrimental for chronically implanted neurostimulation devices.

Pt corrosion can have detrimental effects on the functional lifetime of a chronically implanted neural interface device by altering the geometry, material, and/or electrical properties of its Pt microelectrode(s). Moreover, the byproduct of Pt corrosion may be toxic to the surrounding neural tissue. A Pt concentration as low as 1 ppm is known to cause morphological and functional changes in neurons, and Pt concentrations over 50 ppm are thought to have cytotoxic effects. More recently, evidence has suggested that released Pt during neurostimulation may significantly reduce mitochondrial activity and induce oxidative stress on cells.

Pt corrosion is thought to occur even at low current levels. For example, a Pt corrosion rate of 0.5 μg cm⁻² in vivo for 1.1-mm-diameter circular electrodes is possible even with a low charge density of 20 μC cm⁻². With smaller microelectrodes, the corrosion process is expected to be accelerated. This may especially be problematic for fractal microelectrodes that are thought to have superior charge transfer capabilities relative to conventional circular electrodes. Although the corrosion rate is known to be slower in vivo due to protein layer adsorption on the microelectrodes, the fractal designs are still expected to experience significant corrosion during neurostimulation due to their higher current density.

With the growing demand for more advanced neural interface devices and the increase in the number of neurological disorders capable of being treated with neurostimulation devices, the use of high-density Pt microelectrodes in neurostimulation devices is likely to experience continued growth in the near future. However, the concerns for neural interface stability due to the corrosion of Pt microelectrodes may temper the progress for these advanced microfabricated devices.

In view of the above, it can be appreciated that it would be desirable to reduce or eliminate corrosion of Pt electrodes, including those used as microelectrodes of neurostimulation devices, so as to enable such devices to reduce health risks and remain functional for long-term usage if chronically implanted.

### BRIEF DESCRIPTION OF THE INVENTION

The present invention provides electronic devices that comprise one or more platinum-based electrodes with a protective layer thereon, and provides methods of producing the same.

According to one aspect of the invention, an electronic device includes a platinum-based electrode having a protective layer thereon that includes graphene in an amount effective to reduce platinum corrosion of the electrode.

According to another aspect of the invention, a method of producing an electrode is provided that includes providing a platinum-based electrode on a substrate, and applying a protective layer comprising graphene on the electrode.

According to another aspect of the invention, a method is provided for producing an electrode for a neurostimulation device configured to induce therapeutic neuromodulation of neural circuitry in a subject. The method includes providing a platinum-based electrode on a substrate, and applying a protective layer comprising at least one layer of graphene on the electrode.

Technical effects of devices and methods as described above preferably include the ability to reduce corrosion of platinum-based electrodes during use.

Other aspects and advantages of this invention will be appreciated from the following detailed description.

### BRIEF DESCRIPTION OF THE DRAWINGS

FIGS. 1A and 1B include images showing graphene-coated Pt (G-Pt) microelectrodes with different shapes. FIG. 1A represents a fabrication process of G-Pt microelectrodes that includes metal patterning for microelectrodes and contact pads on silicon oxide on a silicon wafer. The illustrated process represents the steps of transferring a monolayer of graphene, graphene patterning for microelectrode sites, and SU-8 patterning for the passivation layer. FIG. 1B shows the fabricated G-Pt microelectrodes. Scale bar=100 μm.

FIGS. 2A and 2B represent Pt microelectrodes corrosion. FIG. 2A shows a Pt microelectrode with the fractal design before (top) and after (bottom) a three day stimulation. Scale bar=50 μm. FIG. 2B shows a circular Pt microelectrodes before (top) and after (bottom) a three day stimulation. Scale bar=50 μm.

FIG. 3A represents Pt concentration in a phosphate-buffered saline solution (PBS) from the fractal and circle microelectrodes with Pt and G-Pt. FIG. 3B represents total Pt dissolution for ten hours of stimulation, which showed statistically significant reduction for both fractal and circular microelectrodes (* for p<0.05, and ** for p<0.01).

FIGS. 4A through 4E represent Cyclic voltammetry (CV) measurements of Pt and G-Pt microelectrodes. FIG. 4A represents the CV of fractal Pt microelectrodes before and after the stimulation. FIG. 4B represents the CV of the circular Pt microelectrodes. FIG. 4C represents CV measurements on the fractal G-Pt microelectrodes. FIG. 4D represents CV measurements on the circular G-Pt microelectrodes. FIG. 4E represents charge storage capacity of each microelectrode (n=5 for each). Note that ANOVA showed statistically significant differences between microelectrodes (**, p<0.01).

FIGS. 5A through 5G represent measurements of electrochemical impedance spectroscopy. FIG. 5A represents Bode plots of the bare Pt microelectrodes with different shapes before and after the stimulation. FIG. 5B represents Bode plots of the G-Pt microelectrodes. FIG. 5C represents impedance of Pt microelectrodes at 1 kHz (* for p<0.05, and ** for p<0.01). FIG. 5D represents impedance of G-Pt at 1 kHz. FIG. 5E represents Nyquist plots of the bare Pt microelectrodes. FIG. 5F represents Nyquist plots of the G-Pt microelectrodes. FIG. 5G represents an equivalent circuit model for each microelectrode in PBS with BSA.

FIGS. 6A through 6F represent voltage transients measurements. FIG. 6A represents voltage transient of a microelectrode with biphasic, symmetrical current pulse at 50 Hz frequency. FIG. 6B represents voltage transients from Pt microelectrodes with circular and fractal shape before and after a ten-hour stimulation. FIG. 6C represents voltage transients from G-Pt microelectrodes with circular and fractal shapes before and after ten hours of stimulation. FIG. 6D represents maximum negative potential excursion. FIG. 6E represents driving voltage from the microelectrodes. FIG. 6F represents charge injection limit (* for p<0.05, and ** for p<0.01).

### DETAILED DESCRIPTION OF THE INVENTION

Disclosed herein are electronic devices, such as neurostimulation devices, and methods of fabricating the same, and protective layers for protecting electrodes of such devices. More particularly, such a protective layer comprises or consists entirely of one or more layers of graphene that significantly reduces or eliminates the corrosion of an electrode, such as a platinum-based microelectrode, while maintaining good charge transfer characteristics, particularly over an extend period of time, as a nonlimiting example, during prolonged neurostimulation performed with a neurostimulation device.

In experimental investigations discussed below, monolayers of graphene were investigated as protective layers for electrodes formed entirely of platinum. The invention is not limited to electrodes formed entirely of platinum, and instead generally encompasses the use of platinum-based (Pt-based) electrodes, which as used herein refers to electrodes that may be formed entirely of platinum or formed entirely of a platinum alloy whose dominant constituent is platinum (including but not limited to a platinum-iridium alloy) as well as electrodes having at least an exposed outer surface formed entirely of platinum or formed entirely of a platinum alloy whose dominant constituent is platinum (including but not limited to a platinum-iridium alloy). The term “monolayer” is used in the ordinary sense as a single, closely-packed layer of atoms that may be referred to as a 2D material, and a graphene monolayer is understood to refer to a two-dimensional carbon sheet having a honeycomb structure. While protective layers consisting of a single monolayer of graphene were evaluated during the experimental investigations discussed below, protective layers comprising one or more monolayers of graphene or formed entirely of multiple monolayers (multilayers) of graphene are also within the scope of this invention.

The experimental investigations included the microfabrication and testing of fractal and circular Pt microelectrodes to measure their corrosion rates during a prolonged neurostimulation in a proteinaceous buffer solution. Corrosion rates of the bare (uncoated) Pt microelectrodes were compared with that of graphene-coated Pt (G-Pt) microelectrodes using an inductively coupled plasma-mass spectroscopy (ICP-MS), and compositional changes were observed using an X-ray energy dispersive spectroscopy (EDX). Furthermore, changes in electrochemical properties of various microelectrodes were measured before and after an extended neurostimulation. It was observed that a graphene monolayer significantly decreased the Pt corrosion rate to negligible levels even for fractal microelectrodes without any notable reduction in charge transfer characteristics. These results suggest that a graphene monolayer may be used to virtually eliminate Pt-corrosion in chronically implantable neural interface devices. Moreover, these results suggested a path forward for utilizing the fractal microelectrodes for high-density neural stimulation applications (e.g., deep brain stimulation, vision prostheses, etc.) without the potential reliability and health risk issues previously noted.

Nonlimiting embodiments of the invention will now be described in reference to the experimental investigations.

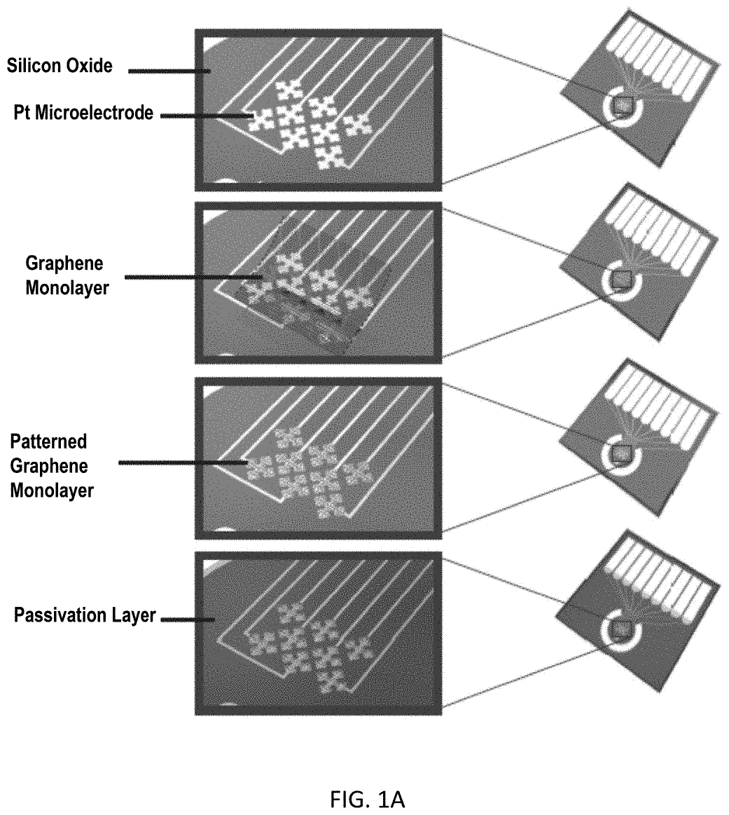

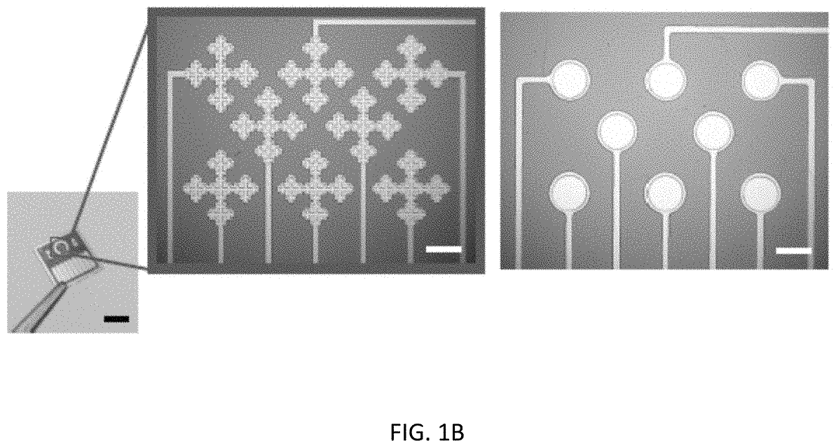

As noted above, G-Pt microelectrodes with the Vicsek fractal shape and circular microelectrodes were fabricated. The fractal microelectrodes were configured to have the same surface area as the circular microelectrodes (about 7854 μm²). FIG. 1A represents the overall fabrication flow. Specifically, a graphene monolayer was grown on a copper (Cu) substrate by low pressure chemical vapor deposition (CVD) and was transferred onto Pt microelectrodes using wet graphene transfer. Graphene was then patterned using a reactive ion etcher (RIE), which was subsequently passivated and patterned using SU-8 (epoxy-based photoresist) leaving only the microelectrodes and contact pads exposed. Bare (uncoated) Pt microelectrodes having the same fractal and circular designs were also fabricated for comparison.

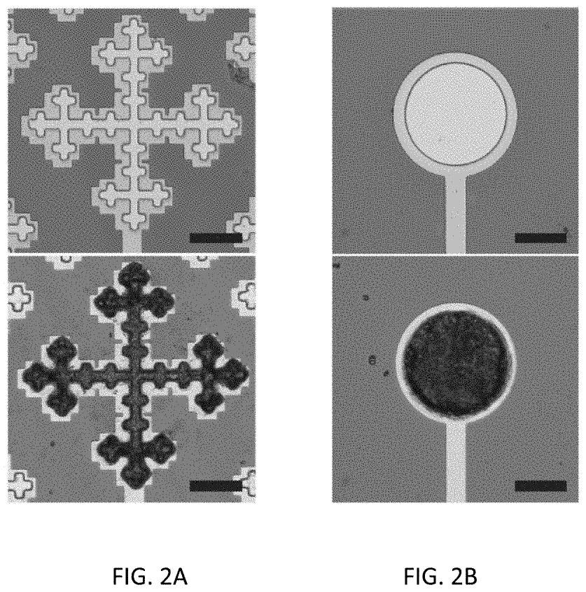

FIGS. 2A and 2B show bare Pt microelectrodes before and after a continuous three-day stimulation using 0.35 mC cm′ at 50 Hz, which is below the safety charge injection limit for Pt microelectrodes. Both fractal and circular designs showed significant corrosion only after three days in a proteinaceous phosphate-buffered saline solution (PBS).

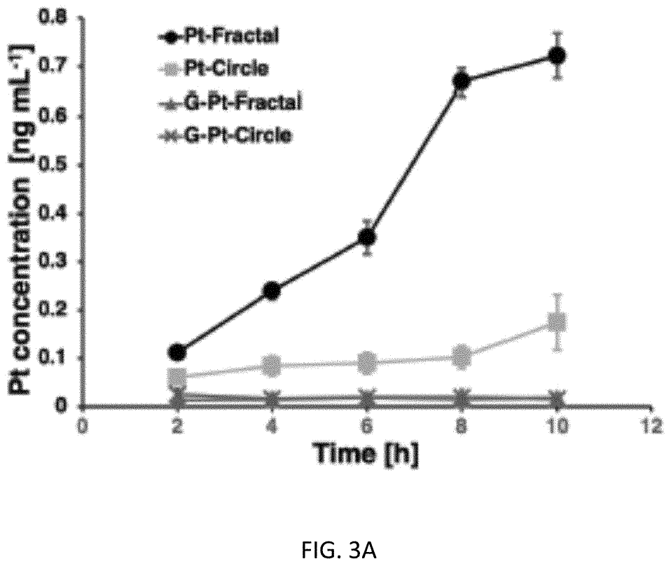

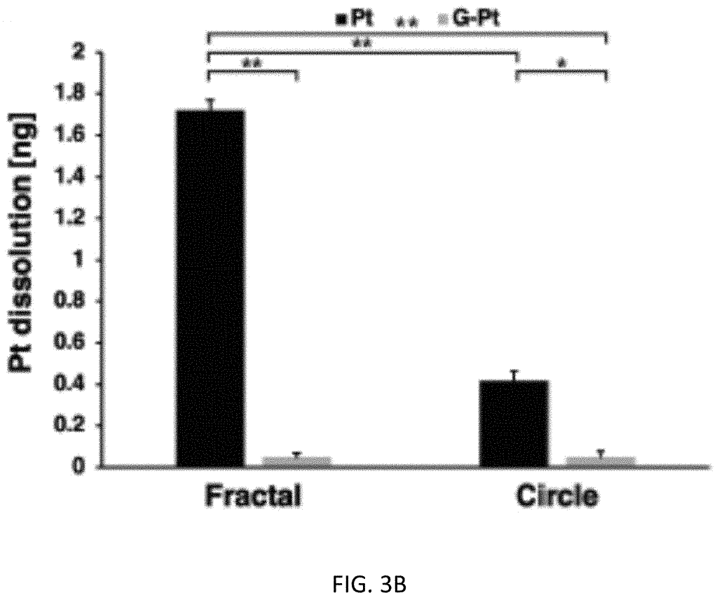

To measure the corrosion rate, the PBS was sampled every two hours during the ten-hour stimulation of each microelectrode type and the Pt concentration change was measured using inductively coupled plasma mass spectrometry (ICP-MS) (n=3, each). FIG. 3A compares the amount of Pt released over the stimulation period for bare and G-Pt microelectrodes with circular and fractal designs. As represented, the bare Pt microelectrodes with fractal design showed the highest corrosion rate with 35.4 ng C⁻¹ and its circular counterpart had a dissolution rate of a 8.7 ng C⁻¹ for 10-hour stimulation. Conversely, both fractal and circular G-Pt microelectrodes exhibited significant reduction in Pt corrosion rate compared to their bare Pt counterparts (1.0 ng C⁻¹ for both), which indicated that the graphene monolayer effectively inhibited corrosion as a diffusion barrier.

When comparing the total amount of Pt lost due to corrosion, the effectiveness of the graphene monolayer in preventing corrosion became clearer (FIG. 3B). For fractal microelectrodes, the graphene layer reduced Pt corrosion by about 97% after ten hours (p<0.01). For circular microelectrodes, it reduced Pt corrosion by about 88% (p<0.01). For a longer stimulation period, it is expected that the percent reduction may be even larger for each microelectrode design. To explore the stability of the graphene layer on a Pt microelectrode surface, Raman spectroscopy was performed on surface of a G-Pt microelectrode. The characteristic peaks for the graphene monolayer were observed both before and after the neurostimulation, which suggested that the graphene layer was not affected by the prolonged biphasic electrical stimulation. Compositional changes were further confirmed using energy-dispersive X-ray spectroscopy (EDX). After ten hours of stimulation, both fractal and circular bare Pt microelectrodes had higher oxygen and lower Pt contents than before the stimulation. In contrast, little change was observed in oxygen and Pt contents on G-Pt microelectrodes following the ten-hour stimulation.

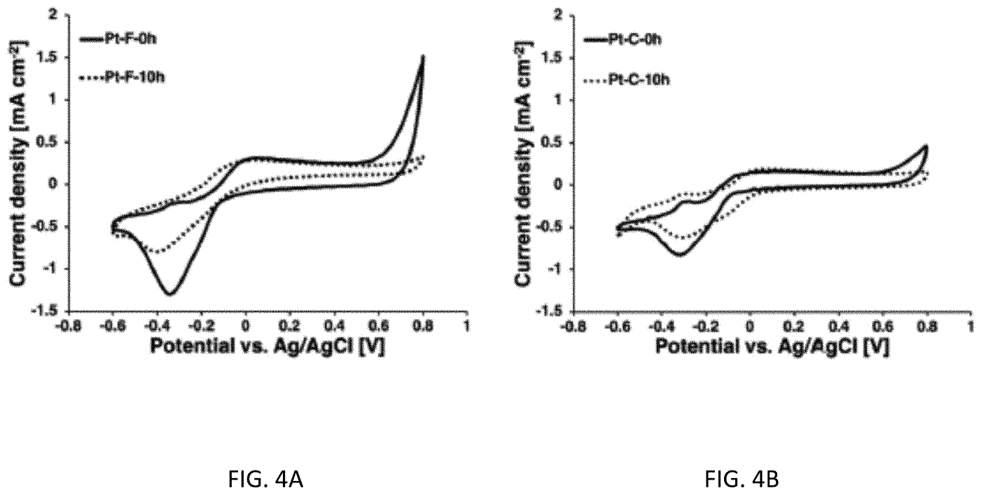

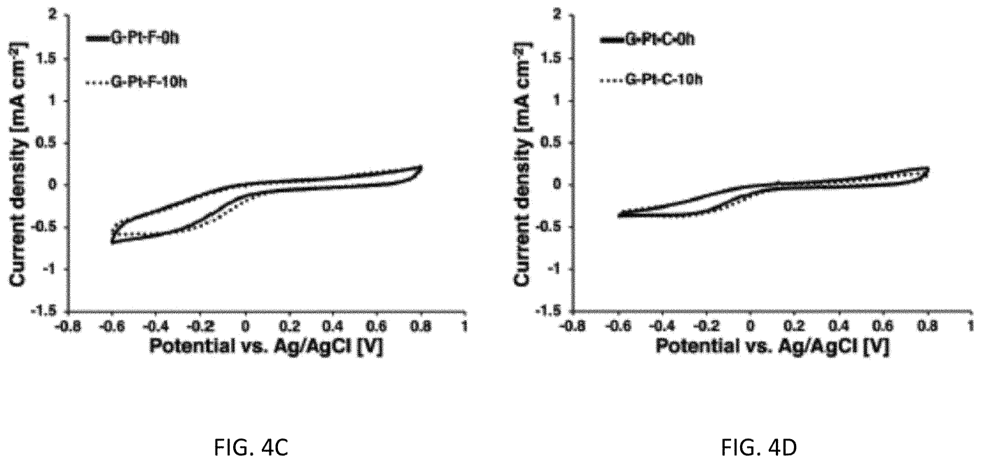

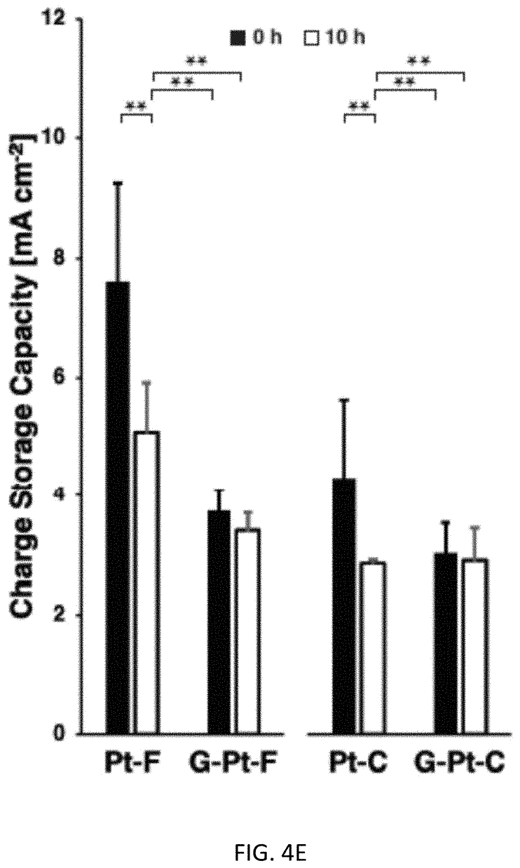

To investigate the impact of Pt corrosion on the charge storage capacities (CSC) of Pt-based microelectrodes, Cyclic voltammetry (CV) measurements were performed on bare Pt and G-Pt microelectrodes with different designs. CV were recorded from −0.6 V to 0.8 V with a scan rate of 50 mV s⁻¹. FIGS. 4A and 4B show a substantial decrease in oxidation and reduction peaks following ten hours of stimulation using bare Pt microelectrodes with either fractal or circular designs. However, G-Pt microelectrodes demonstrated little change in CV after the same treatment (FIGS. 4C and 4D). These results suggested that the bare Pt microelectrodes not only demonstrated physical changes (FIG. 2) but they also underwent substantial changes to their electrochemical characteristics after only ten hours of continuous stimulation. Moreover, this suggested that a graphene layer can protect a Pt surface from corrosion and prevent changes in charge transfer characteristics.

The CSC measures the total amount of charge available for a single stimulation pulse, which is an indication of microelectrode charge injection capacity. The CSC was calculated using the following:

$$CSC = \frac{1}{vA} {\int }_{{E}_{c}}^{{E}_{a}} \vert i \vert dE ( C \text{/} {cm}^{2} ) \qquad ( 1 )$$

with the potential versus Ag/AgCl reference electrode E, the measured current I, the positive and negative potential boundary Eₐ and E_{c}, the surface area of the microelectrode A, and the scan rate v. The CSC for each microelectrode before and after the ten-hour stimulation were compared using one-way analysis of variance (ANOVA) with Tukey's HSD post-hoc test. The results showed that CSC of bare Pt microelectrodes decreased significantly after the ten-hour stimulation (p<0.01). As expected, the fractal microelectrodes showed a larger CSC decrease than the circular microelectrodes. However, no statistically significance differences were observed between CSC of G-Pt microelectrodes following the stimulation for either fractal or circular designs, which further evidenced the Pt corrosion prevention properties of graphene.

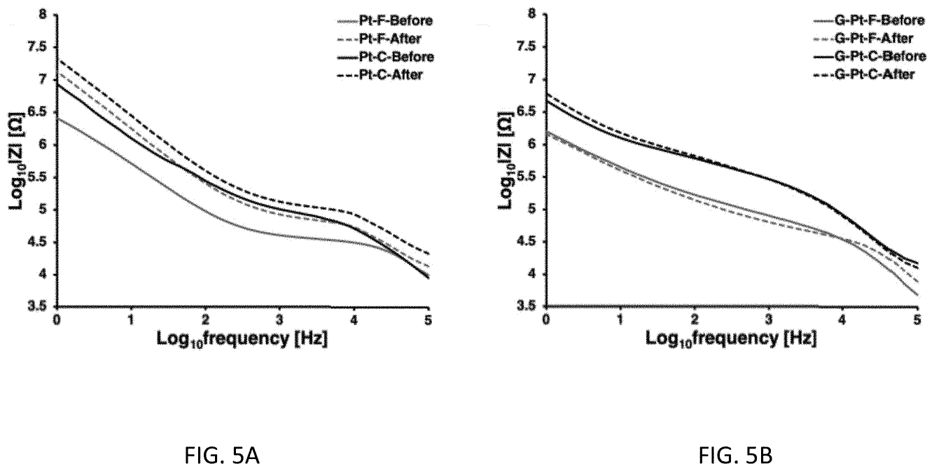

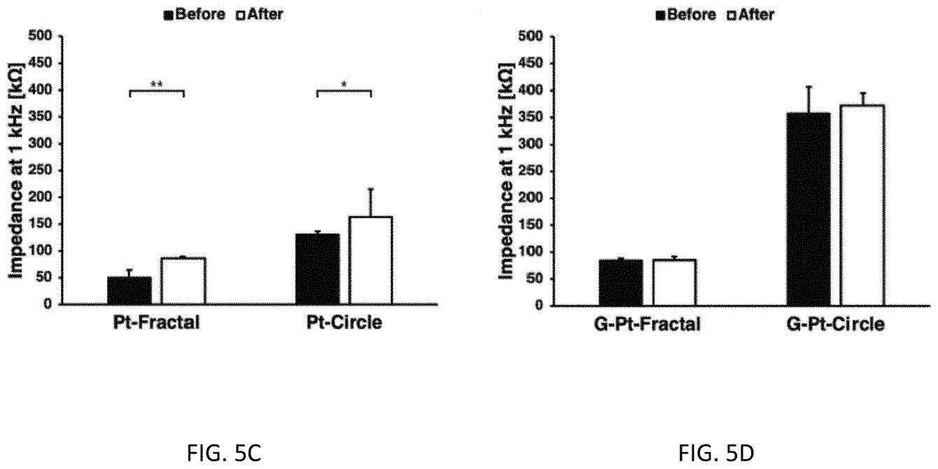

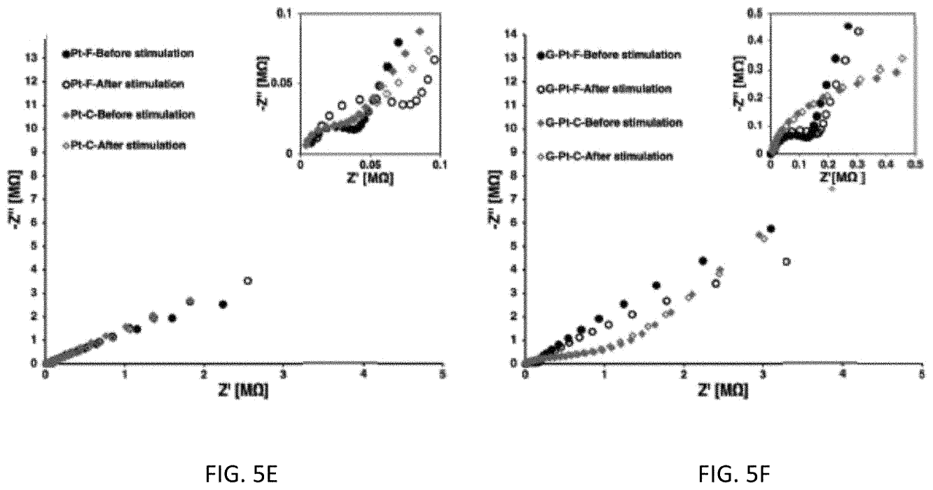

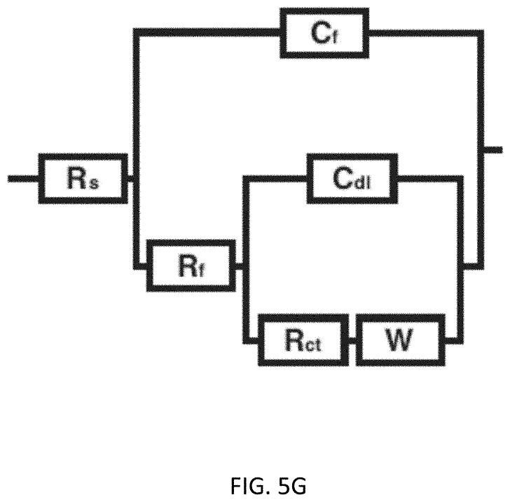

Electrochemical impedance spectroscopy (EIS) was performed to monitor the changes in microelectrode impedance following the stimulation (n=5, each). FIG. 5A shows the impedance spectra of the bare Pt and G-Pt microelectrodes before and after the stimulation. Throughout the entire frequency range, the impedance of bare Pt microelectrodes increased (FIG. 5A). In contrast, relatively small differences occurred in the G-Pt microelectrodes (FIG. 5B). When comparing the impedance at 1 kHz, the impedance of bare Pt microelectrodes increased significantly following the stimulation (FIG. 5C). Conversely, no significant differences in impedances were observed for G-Pt microelectrodes after the stimulation period (FIG. 5D).

FIGS. 5E and 5F show representative Nyquist plots for each microelectrode type fitted to an equivalent circuit model to estimate the parameters of a solution resistance (Rₛ), a charge transfer resistance (R_{ct}), a double layer capacitance (C_{dl}), a resistance of the adsorbed protein film (R_{f}), capacitance of the protein film (C_{f}), and the Warburg element (W) (FIG. 5G). The estimated parameters from bare Pt microelectrodes show that the solution resistances, double layer capacitances, and protein film capacitance of the microelectrodes decreased whereas W increased after ten hours of stimulation. The changes were more pronounced for fractal than circular microelectrodes, which highlight the risk of using unprotected fractal microelectrodes. However, G-Pt microelectrodes showed minimal changes across all estimated EIS parameters, which further supported the conclusion that graphene is capable of performing as a protective layer and prolong the lifetime of fractal microelectrodes.

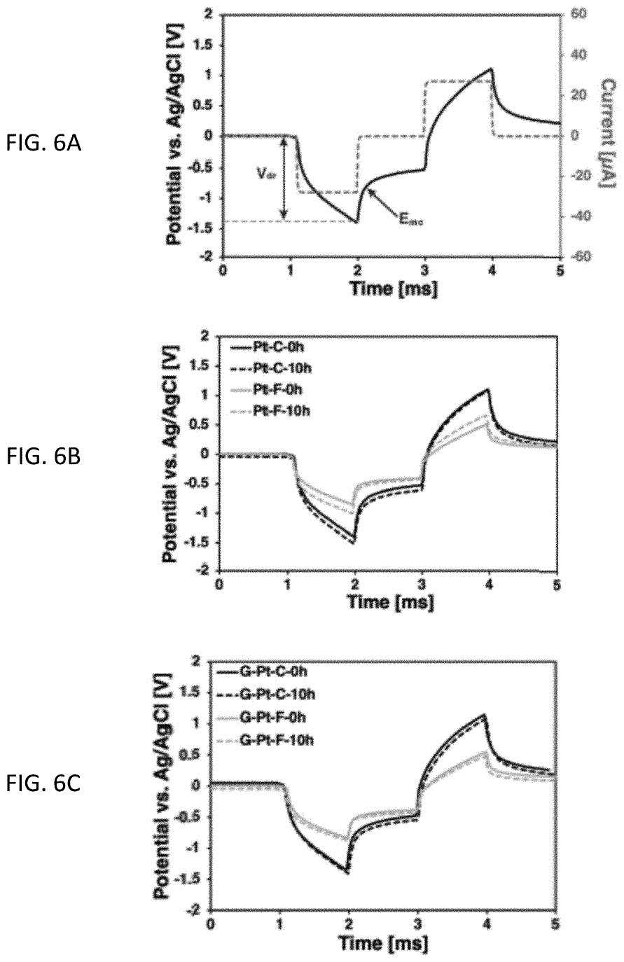

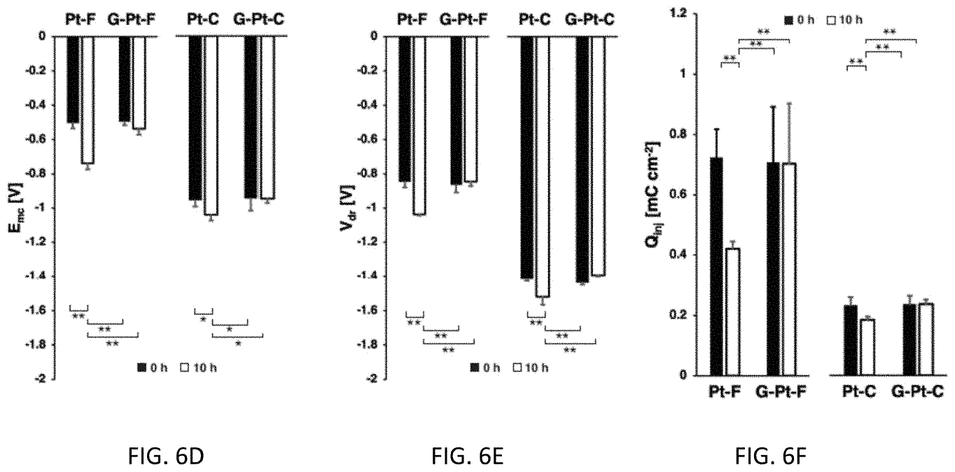

The voltage transient characteristics of the microelectrodes were compared to confirm the long-term stimulation charge-injection capacity (n=5, each). Each microelectrode was stimulated using biphasic, symmetric pulses with 1 ms pulse width at 26.97 nC per phase (0.35 mC cm⁻² with 26.97 μA at 50 Hz. The interphase potential was set to 0 V versus Ag/AgCl reference electrode. To compare, the maximum negative potential excursion (E_{mc}), the maximum driving voltage (V_{dr}), and the charge injection limit (Qᵢₙⱼ) from the voltage transient responses were measured (FIG. 6A). FIG. 6B shows that the maximum negative voltages of both types of bare Pt microelectrodes increased after the ten hours of stimulation. However, G-Pt microelectrodes maintained relatively stable voltage transient responses following the stimulation (FIG. 6C).

The E_{mc} is the potential required to polarize the microelectrode, which is measured at the end of the cathodic phase of the biphasic pulse. FIGS. 6D and 6E show the comparison of E_{mc} and V_{dr} for each microelectrode at 26.97 nC per phase. In general, fractal microelectrodes have lower E_{mc} and V_{dr} than the circular ones. Moreover, the bare Pt fractal microelectrodes showed a larger increase in E_{mc} and V_{dr} following ten hours of stimulation than the circular microelectrodes, which highlight the design's vulnerability. However, G-Pt microelectrodes showed virtually no change in E_{mc}, and V_{dr} following the stimulation.

When comparing the Qᵢₙⱼ of each microelectrode, the benefit of G-Pt became even more apparent (FIG. 6E). The results showed that bare fractal microelectrodes suffered significant loss in Qᵢₙⱼ after a ten-hour stimulation while G-Pt microelectrodes maintained its Qᵢₙⱼ. This bodes well for the high performing fractal designs because their post-stimulation Qᵢₙⱼ remained greater than three times that of the circular microelectrodes.

These investigations indicated that long-term stimulation of Pt microelectrodes can result in corrosion-induced electrode degradation and failure, fractal microelectrodes have significantly superior charge transfer characteristics than simple circular design, and fractal microelectrodes are more susceptible to stimulation-induced corrosion. However, these results also indicated that a graphene monolayer can significantly reduce the stimulation-induced corrosion in Pt microelectrodes. Taken together, the results suggested that G-Pt fractal microelectrodes may provide a more reliable method of interfacing with neural substrates.

The following paragraphs provide additional details on the above-described experimental investigations.

Arrays of platinum microelectrodes were fabricated on 500 nm film of silicon oxide grown by thermal oxidation of a silicon wafer, though various other substrate materials may be used, such as but not limited to silicon, silicon nitride, parylene, polyimide, etc. Microelectrodes patterns were defined using a positive photoresist (AZ1518, MicroChem, Newton, Mass., USA), which was followed by deposition of a Ti adhesion layer (10 nm) and a Pt layer (100-nm thick) using an e-beam evaporator, though various other deposition processes may be used, as nonlimiting examples, chemical vapor deposition, physical vapor deposition, plasma enhanced deposition, electrochemical, etc. Furthermore, other materials may be used as the adhesion layer, as nonlimiting examples, Au, Cr/Ni, etc. The metal patterns were achieved by a lift-off process using acetone. An SU-8 passivation layer (1.5 μm thick) was spin-coated and patterned using photolithography.

To fabricate the G-Pt microelectrodes, the monolayer of graphene was grown on Cu substrate by LPCVD at 1000° C. using methane as carbon precursor. Polymethyl methacrylate (PMMA) was first spin coated on the graphene layer to aid the transfer process. After curing the PMMA at 180° C. for 5 min, the Cu was etched away by FeCl₃ solution. The PMMA/graphene stack was washed with deionized water, then the stack was transferred onto Pt patterned substrate. PMMA was removed using acetone, the sample was cleaned with isopropyl alcohol. The transferred graphene was patterned using photolithography and reactive ion etching with oxygen plasma. Finally, SU-8 was coated and patterned for passivation layer.

For inductively coupled plasma mass spectrometry (ICP-MS) analysis, aliquots of the PBS in the testing chamber were taken every 2 hours for 10 hours. Collected samples were digested using aqua regia and diluted into 4% HCl for the ICP-MS analysis. ICP-MS analysis was performed using Thermo Element II ICP-MS ((ThermoFisher Scientific, Waltham, Mass., USA).

Cyclic voltammetry and electrochemical impedance spectroscopy was measured using a potentiostat (SP-200, Bio-Logic. Inc, Seyssinet-Pariset, France) with Ag/AgCl with 3M KCl (RE-1CP, ALS Co., Ltd, Tokyo, Japan), graphite counter electrode, and working electrodes on the microelectrode array. CV was measured in a PBS with composition of 1.1 mM KH₂PO₄, 155 mM NaCl, 3 mM Na₂HPO₄.H₂O with pH 7.4 (ThermoFisher Scientific, Waltham, Mass., USA). Bovine serum albumin (0.2 mg/ml, BSA, ThermoFisher Scientific, Waltham, Mass., USA) was added to PBS. Scan rate for CV was 50 mV between potential range of −0.65 V and 0.85 V versus Ag/AgCl reference electrode, which is the water window of Pt. EIS were measured with the AC voltage perturbation potential of 30 mV amplitude in the frequency range from 1 to 100 kHz in PBS with BSA.

To measure voltage transient with long-term stimulation, the charge-balanced biphasic current pulse was applied using the sourcemeter (2601A, Keith-ley, Cleveland, Ohio, USA). The pulsing was done at 50 Hz with a 1 ms pulse width and 1 ms inter-phase delay. The current pulses were injected into the microelectrode, and a data acquisition board (NI USB-6333, National Instruments, Austin, Tex., USA) was used to record the voltage transient. The time delay that the applied current is completely off was measured to be approximately 50 μs, therefore, E_{mc}, was estimated at 50 us immediately after the end of the cathodic pulse. To estimate Qᵢₙⱼ, E_{mc} of each microelectrode was measured in the range of specific injected charge density (15, 20, 25, 30, 35 mA cm⁻²). Regression function was estimated using the E_{mc} points in the injected charge density range, and Qᵢₙⱼ was calculated by the regression function.

While the invention has been described in terms of specific or particular embodiments and investigations, it should be apparent that alternatives could be adopted by one skilled in the art. For example, the electrodes could differ in size, shape, material, appearance, and construction from the embodiments described herein and shown in the drawings, the electrodes may be used in various devices, process parameters such as temperatures and durations could be modified, and appropriate materials could be substituted for those noted. As a nonlimiting example, though the experimental investigations involved the microfabrication and testing of fractal and circular microelectrodes, the microelectrodes could have essentially any geometry, including but not limited to Euclidean (for example, rectangular, etc.) geometries and non-Euclidean (for example, serpentine, irregular, asymmetric, etc.) geometries. Accordingly, it should be understood that the invention is not necessarily limited to any embodiment described herein or illustrated in the drawings. It should also be understood that the phraseology and terminology employed above are for the purpose of describing the disclosed embodiments and investigations, and do not necessarily serve as limitations to the scope of the invention. Therefore, the scope of the invention is to be limited only by the following claims.

1. An electronic device comprising a platinum-based electrode having a protective layer thereon comprising graphene in an amount effective to reduce platinum corrosion of the electrode.

2. The electronic device of claim 1, wherein at least an exposed outer surface of the platinum-based electrode is formed entirely of platinum or formed entirely of a platinum-iridium alloy.

3. The electronic device of claim 1, wherein the protective layer comprises a monolayer of graphene.

4. The electronic device of claim 1, wherein the protective layer consists of at least one monolayer of graphene.

5. The electronic device of claim 1, wherein the electronic device is a neurostimulation device configured to induce therapeutic neuromodulation of neural circuitry in a subject.

6. The electronic device of claim 5, wherein the neurostimulation device is an invasive (implantable) device or a noninvasive device.

7. A method comprising chronically implanting the neurostimulation device of claim 5 in the subject to target subcortical, cortical, spinal, cranial, or peripheral nerve structures, modulate neuronal activity, and provide a therapeutic effect for a neuropsychiatric disorder.

8. A method of producing an electrode of an electronic device, the method comprising:
providing a platinum-based electrode on a surface of a substrate; and
applying a protective layer comprising graphene on the electrode.

9. The method of claim 8, wherein the providing step comprises depositing an adhesion layer on the substrate and then depositing platinum on the adhesion layer such that the adhesion layer is located between the substrate and the platinum.

10. The method of claim 8, wherein at least an exposed outer surface of the platinum-based electrode is formed entirely of platinum or formed entirely of a platinum-iridium alloy.

11. The method of claim 8, wherein the protective layer comprises a monolayer of graphene.

12. The method of claim 8, wherein the protective layer consists of at least one monolayer of graphene.

13. The method of claim 8, wherein the electronic device is a neurostimulation device configured to induce therapeutic neuromodulation of neural circuitry in a subject.

14. The method of claim 8, further comprising producing the protective layer by:
growing a monolayer of graphene on a substrate;
removing the monolayer of graphene from the substrate; and
transferring the monolayer of graphene onto the electrode.

15. The method of claim 14, wherein the monolayer of graphene is grown on the substrate via low pressure chemical vapor deposition.

16. The method of claim 14, wherein the substrate is removed from the monolayer of graphene via chemical etching.

17. The method of claim 14, wherein the monolayer of graphene is transferred onto the electrode via wet graphene transfer.

18. The method of claim 14, further comprising:
applying a transfer assist coating onto the monolayer of graphene prior to transfer thereof onto the electrode;
transferring the monolayer of graphene with the transfer assist coating thereon onto the electrode; and
removing the transfer assist coating.

19. A method of producing an electrode for a neurostimulation device configured to induce therapeutic neuromodulation of neural circuitry in a subject, the method comprising:
providing a platinum-based electrode on a substrate; and
applying a protective layer comprising a monolayer of graphene on the electrode.
